(19) United States
(12) Patent Application Publication
(10) Pub. No.: US 2008/0255213 A1
(43) Pub. Date: Oct. 16, 2008

(54) THIAZOLIDINONE AMIDES, THIAZOLIDINE CARBOXYLIC ACID AMIDES, AND SERINE AMIDES, INCLUDING POLYAMINE CONJUGATES THEREOF, AS SELECTIVE ANTI-CANCER AGENTS

(75) Inventors: Duane D. Miller, Germantown, TN (US); James T. Dalton, Columbus, OH (US); Wei Li, Germantown, TN (US); Lu Yan, Bartlett, TN (US)

Correspondence Address: NIXON PEABODY LLP - PATENT GROUP, 1100 CLINTON SQUARE, ROCHESTER, NY, 14604, US

(73) Assignees: University of Tennessee Research Foundation, Knoxville, TN (US); Ohio State University Research Foundation, Columbus, OH (US)

(21) Appl. No.: 12/102,575

(22) Filed: Apr. 14, 2008

Related U.S. Application Data
(60) Provisional application No. 60/911,882, filed on Apr. 14, 2007.

(51) Int. Cl. A61K 31/426; C07D 277/04; C12N 5/00; A61P 35/00

(52) U.S. Cl. 514/365; 548/200; 435/375

(57) ABSTRACT

Substituted thiazolidinone carboxylic acid amides and substituted thiazolidine carboxylic acid amides having a structure

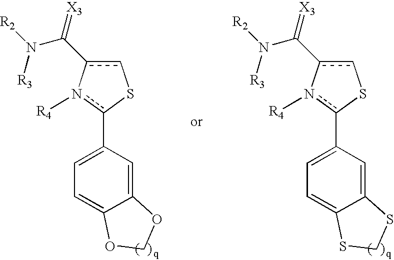

where the various substituent groups are as defined in the specification. Methods of making these compounds, pharmaceutical compositions containing the compounds, and their use, particularly for treating or preventing cancer, are also disclosed.

This application claims the priority benefit of U.S. Provisional Patent Application Ser. No. 60/911,882, filed Apr. 14, 2007, which is hereby incorporated by reference in its entirety.

This invention was made with funding received from the U.S. Department of Defense under grant DAMD 17-01-1-0830. The U.S. government has certain rights in this invention.

### FIELD OF THE INVENTION

The present invention relates to novel thiazolidinone amides, novel thiazolidine carboxylic acid amides, methods of making these compounds, and uses thereof, particularly for treating various cancers including but not limited to prostate, breast, ovarian, and skin cancers.

### BACKGROUND OF THE INVENTION

Prostate cancer accounts for 33% of all newly diagnosed malignancies among men in the United States (American Cancer Society: Cancer Facts and Figures (2003)). According to the American Cancer Society, an estimated 230,110 men will be diagnosed with prostate cancer in 2004, and 29,900 men will die of it (American Cancer Society: Cancer Facts and Figures (2004)). The incidence of prostate cancer varies worldwide, with the highest rates found in the United States, Canada, and Scandinavia, and the lowest rates found in China and other parts of Asia (Quinn and Babb, “Patterns and Trends in Prostate Cancer Incidence, Survival, Prevalence and Mortality. Part: International Comparisons,” BJU Int. 90:162-173 (2002); Gronberg, “Prostate Cancer Epidemiology,” Lancet 361:859-864 (2003)). These differences are caused by genetic susceptibility, exposure to unknown external risk factors, differences in health care and cancer registration, or a combination of these factors.

Cancer of the prostate is multifocal and it is commonly observed that the cancerous gland contains multiple independent lesions, suggesting the heterogeneity of the disease (Foster et al., “Cellular and Molecular Pathology of Prostate Cancer Precursors,” Scand. J. Urol. Nephrol. 205:19-43 (2000)). Determinants responsible for the pathologic growth of the prostate remain poorly understood, although steroidal androgens and peptide growth factors have been implicated (Agus et al., “Prostate Cancer Cell Cycle Regulators: Response to Androgen Withdrawal and Development of Androgen Independence,” J. Natl. Cancer. Inst. 91:1869-1876 (1999); Djakiew, “Dysregulated Expression of Growth Factors and Their Receptors in the Development of Prostate Cancer,” Prostate 42:150-160 (2000)). As long as the cancer is confined to the prostate, it can be successfully controlled by surgery or radiation, but in metastatic disease, few options are available beyond androgen ablation (Frydenberg et al., “Prostate Cancer Diagnosis and Management,” Lancet 349:1681-1687 (1997)), the mainstay of treatment in the case of lymph node involvement or disseminated loci. Once tumor cells have become hormone refractory, the standard cytotoxic agents are marginally effective in slowing disease progression, although they do provide some degree of palliative relief. Current chemotherapeutic regimens, typically two or more agents, afford response rates in the range of only 20-30% (Beedassy et al., “Chemotherapy in Advanced Prostate Cancer,” Sem. Oncol. 26:428-438 (1999); Raghavan et al., “Evolving Strategies of Cytotoxic Chemotherapy for Advanced Prostate Cancer,” Eur. J. Cancer 33:566-574 (1997)).

One promising drug development strategy for prostate cancer involves identifying and testing agents that interfere with growth factors and other molecules involved in the cancer cell's signaling pathways. G-protein coupled receptors (“GPCRs”) are a family of membrane-bound proteins that are involved in the proliferation and survival of prostate cancer cells initiated by binding of lysophospholipids (“LPLs”) (Raj et al., “Guanosine Phosphate Binding Protein Coupled Receptors in Prostate Cancer: A Review,” J. Urol. 167:1458-1463 (2002); Kue et al., “Essential Role for G Proteins in Prostate Cancer Cell Growth and Signaling,” J. Urol. 164:2162-2167 (2000); Guo et al., “Mitogenic Signaling in Androgen Sensitive and Insensitive Prostate Cancer Cell Lines,” J. Urol. 163:1027-1032 (2000); Barki-Harrington et al., “Bradykinin Induced Mitogenesis of Androgen Independent Prostate Cancer Cells,” J. Urol. 165:2121-2125 (2001)). The importance of G protein-dependent pathways in the regulation of growth and metastasis in vivo is corroborated by the observation that the growth of androgen-independent prostate cancer cells in mice is attenuated by treatment with pertussis toxin, an inhibitor of Gi/o proteins (Bex et al., “Influence of Pertussis Toxin on Local Progression and Metastasis After Orthotopic Implantation of the Human Prostate Cancer Cell Line PC3 in Nude Mice,” Prostate Cancer Prostatic Dis. 2:36-40 (1999)). Lysophosphatidic acid (“LPA”) and sphingosine 1-phosphate (“SIP”) are lipid mediators generated via the regulated breakdown of membrane phospholipids that are known to stimulate GPCR-signaling.

LPL binds to GPCRs encoded by the Edg gene family, collectively referred to as LPL receptors, to exert diverse biological effects. LPA stimulates phospholipase D activity and PC-3 prostate cell proliferation (Qi et al., “Lysophosphatidic Acid Stimulates Phospholipase D Activity and Cell Proliferation in PC-3 Human Prostate Cancer Cells,” J. Cell. Physiol. 174:261-272 (1998)). Further, prior studies have shown that LPA is mitogenic in prostate cancer cells and that PC-3 and DU-145 express LPA₁, LPA₂, and LPA₃ receptors (Daaka, “Mitogenic Action of LPA in Prostate,” Biochim. Biophys. Acta. 1582:265-269 (2002)). Advanced prostate cancers express LPL receptors and depend on phosphatidylinositol 3-kinase (“PI3K”) signaling for growth and progression to androgen independence (Kue and Daaka, “Essential Role for G Proteins in Prostate Cancer Cell Growth and Signaling,” J. Urol. 164:2162-2167 (2000)). Thus, these pathways are widely viewed as one of the most promising new approaches to cancer therapy (Vivanco et al., “The Phosphatidylinositol 3-Kinase AKT Pathway in Human Cancer,” Nat. Rev. Cancer 2:489-501 (2002)) and provide an especially novel approach to the treatment of advanced, androgen-refractory prostate cancer. Despite the promise of this approach, there are no clinically available therapies that selectively exploit or inhibit LPA or PI3K signaling.

Melanoma is the most aggressive form of skin cancer and is the fastest growing cancer currently in the United States (Ries et al., “The Annual Report to the Nation on the Status of Cancer, 1993-1997, with a Special Section on Colorectal Cancer,” Cancer 88:2398-2424 (2000); Jemal et al., “Recent Trends in Cutaneous Melanoma Incidence Among Whites in the United States,” Cancer Inst. 93:678-683 (2001); Jemal et al., “Cancer Statistics, 2004,” CA Cancer J. Clin. 54:8-29 (2004)). It is the most common cancer in young adults aged 20-30. Approximately two to three out of 100,000 people per year die from melanoma in the northern hemisphere (Marks, “Epidemiology in Melanoma,” Clin. Exp. Dermatol. 25:459-463 (2000); Lens et al., “Global Perspectives of Contemporary Epidemiological Trends of Cutaneous Malignant Melanoma,” Br. J. Dermatol. 150:179-185 (2004)). While in situ melanoma (stage 0) can usually be cured surgically, melanoma metastized to major organs (stage IV) is virtually incurable. Patients with advanced melanoma have median survival time of 7.5 months and the estimated five year survival rate is only 5-9% (Barth et al., “Prognostic Factors in 1,521 Melanoma Patients with Distant Metastases,” J. Am. Coll. Surg. 181:193-201 (1995); Buzaid et al., “The Changing Prognosis of Melanoma,” Curr. Oncol. Rep. 2:322-328 (2000); Anderson et al., “Systemic Treatments for Advanced Cutaneous Melanoma,” Oncology (Williston Park) 9:1149-1158, discussion 1163-1144, 1167-1148 (1995)).

Currently, dacarbazine (“DTIC”) is the only U.S. Food and Drug Administration (“FDA”) approved drug to treat advanced melanoma, and it provides complete remission in only two percent of patients (Anderson et al., “Systemic Treatments for Advanced Cutaneous Melanoma,” Oncology (Williston Park) 9:1149-1158, discussion 1163-1144, 1167-1148 (1995); Serrone et al., Dacarbazine-based Chemotherapy for Metastatic Melanoma: Thirty-year Experience Overview,” J. Exp. Clin. Cancer Res. 19:21-34 (2000)). The FDA also approved the use of high-dose interferon alpha-2b (“IFN-α2b”) as adjuvant treatment of patients at high risk of recurrence of melanoma, but a total of four recent Phase III randomized trials failed to detect a survival advantage with the addition of IFN-α2b to DTIC (Lawson, “Choices in Adjuvant Therapy of Melanoma,” Cancer Control 12:236-241 (2005); Bajetta et al., “Multicenter Randomized Trial of Dacarbazine Alone or in Combination with Two Different Doses and Schedules of Interferon alpha-2a in the Treatment of Advanced Melanoma,” J. Clin. Oncol. 12:806-811 (1994); Thomson et al., “Interferon alpha-2a Does Not Improve Response or Survival when Combined with Dacarbazine in Metastatic Malignant Melanoma: Results of a Multi-institutional Australian Randomized Trial,” Melanoma Res. 3:133-138 (1993); Young et al., “Prospective Randomized Comparison of Dacarbazine (DTIC) Versus DTIC Plus Interferon-alpha (IFN-alpha) in Metastatic Melanoma,” Clin. Oncol. (R. Coll. Radiol) 13:458-465 (2001)). Several extensive clinical trials have been conducted in recent years with a variety of cancer drugs or combination of cancer drugs, but they all failed to demonstrate clear effect against advanced melanoma (Lawson, “Choices in Adjuvant Therapy of Melanoma,” Cancer Control 12:236-241 (2005); Mandara et al., “Chemotherapy for Metastatic Melanoma,” Exp. Rev. Anticancer Ther. 6:121-130 (2006); Kaufmann et al., “Temozolomide in Combination with Interferon-alpha Versus Temozolomide Alone in Patients with Advanced Metastatic Melanoma: A Randomized, Phase III, Multicenter Study from the Dermatologic Cooperative Oncology Group,” J. Clin. Oncol. 23(25):9001-9007 (2005)). Therefore, DTIC still remains the gold standard for advanced melanoma despite its very limited efficacy (Eggermont et al., “Re-evaluating the role of Dacarbazine in Metastatic Melanoma: What Have We Learned in 30 Years?” Eur. J. Cancer 40:1825-1836 (2004); Atallah et al., “Treatment of Metastatic Malignant Melanoma,” Curr. Treat Options Oncol. 6:185-193 (2005)). With the rapidly rising incidents reported for melanoma in the United States, clearly there is an urgent need to develop more effective therapeutic agents to combat advanced melanoma.

The present invention is directed to overcoming these and other deficiencies in the prior art.

### SUMMARY OF THE INVENTION

A first aspect of the present invention relates to compounds according to formula (I) and formula (II)

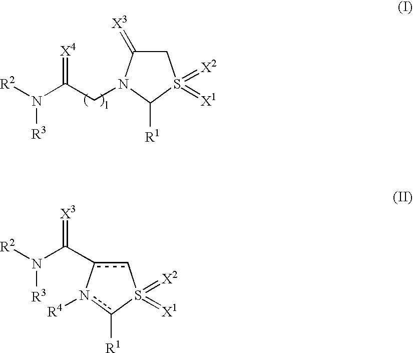

wherein

X¹ and X² are each optional, and each can be oxygen;

X³ and X⁴ are each optional, and each can be oxygen or sulfur;

l is an integer from 1 to 12;

R¹ is selected from the group of saturated or unsaturated cyclic hydrocarbons, saturated or unsaturated N-heterocycles, saturated or unsaturated O-heterocycles, saturated or unsaturated S-heterocycles, saturated or unsaturated mixed heterocycles, aliphatic or non-aliphatic straight- or branched-chain C1 to C30 hydrocarbons, or

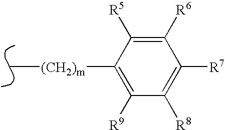

or —(CH₂)ₘ—Y¹ where m is an integer from 0 to 10 and Y¹ is a saturated or unsaturated cyclic hydrocarbon, saturated or unsaturated N-heterocycle, saturated or unsaturated O-heterocycle, saturated or unsaturated S-heterocycle, or saturated or unsaturated mixed heterocycle;

R² is hydrogen, alkoxy, an aliphatic or non-aliphatic straight- or branched-chain C1 to C30 hydrocarbon, R¹⁰—N(Z)-hydrocarbon- or R¹⁰-hydrocarbon- where the hydrocarbon group is an aliphatic or non-aliphatic straight- or branched-chain C1 to C30 hydrocarbon, a saturated or unsaturated cyclic hydrocarbon, a saturated or unsaturated N-heterocycle, a saturated or unsaturated O-heterocycle, a saturated or unsaturated S-heterocycle, a saturated or unsaturated mixed heterocycle,

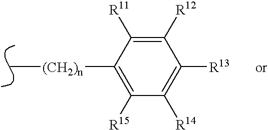

or —(CH₂)ₙ—Y² where n is an integer from 0 to 10 and Y² is a saturated or unsaturated cyclic hydrocarbon, saturated or unsaturated N-heterocycle, saturated or unsaturated O-heterocycle, saturated or unsaturated S-heterocycle, or saturated or unsaturated mixed heterocycle;

R³ is hydrogen, alkoxy, or an aliphatic or non-aliphatic straight- or branched-chain C1 to C10 hydrocarbon;

R⁴ is optional, or can be hydrogen, an aliphatic or non-aliphatic straight- or branched-chain C1 to C10 hydrocarbon, acyl, acetyl, or mesyl;

R⁵, R⁶, R⁷, R⁸, R⁹, R¹⁰, R¹¹, R¹², R¹³, R¹⁴, and R¹⁵ are independently selected from the group of hydrogen, hydroxyl, an aliphatic or non-aliphatic straight- or branched-chain C1 to C10 hydrocarbon, alkoxy, aryloxy, nitro, cyano, chloro, fluoro, bromo, iodo, haloalkyl, dihaloalkyl, trihaloalkyl, amino, alkylamino, dialkylamino, acylamino, arylamino, amido, alkylamido, dialkylamido, arylamido, aryl, C5 to C7 cycloalkyl, and arylalkyl, or any one or more combinations of R⁵ and R⁶, R⁶ and R⁷, R⁷ and R⁸, or R⁸ and R⁹ form a dioxolyl ring (—O—CH₂—O—) or a dioxanyl ring (—O—CH₂—CH₂—O—), or a dithiolanyl ring (—S—CH₂—S—) or a dithianyl ring (—S—CH₂—CH₂—S—) ring;

R¹⁰ is H(Z)N—, H(Z)N-hydrocarbon-, H(Z)N-hydrocarbon-N(Z)-hydrocarbon-, H(Z)N-hydrocarbon-O-hydrocarbon-, hydrocarbon-O-hydrocarbon-, hydrocarbon-N(Z)-hydrocarbon-, H(Z)N-hydrocarbon-carbonyl-hydrocarbon-, hydrocarbon-carbonyl-hydrocarbon-, H(Z)N-phenyl-, H(Z)N-phenylalkyl-, H(Z)N-phenylalkyl-N(Z)-hydrocarbon-, H(Z)N-phenylalkyl-O-hydrocarbon-, phenylalkyl-O-hydrocarbon-, phenylalkyl-N(Z)-hydrocarbon-, H(Z)N-phenylalkyl-carbonyl-hydrocarbon-, or phenylalkyl-carbonyl-hydrocarbon-, wherein each hydrocarbon is independently an aliphatic or non-aliphatic straight- or branched-chain C1 to C10 group, and wherein each alkyl is a C1 to C10 alkyl; and

Z is independently hydrogen or t-butoxycarbonyl.

A second aspect of the present invention relates to a pharmaceutical composition including a pharmaceutically acceptable carrier and a compound according to the first aspect of the present invention.

A third aspect of the present invention relates to a method of destroying a cancer cell that includes the steps of: providing a compound according to the first aspect of the present invention and contacting a cancer cell with the compound under conditions effective to destroy the contacted cancer cell.

A fourth aspect of the present invention relates to a method of treating or preventing a cancerous condition that includes the steps of: providing a compound according to the first aspect of the present invention and administering an amount of the compound to a patient in a manner effective to treat or prevent a cancerous condition.

A fifth aspect of the present invention relates to a method of making a compound according to formula (I) that includes the steps of: reacting an intermediate according to formula (III),

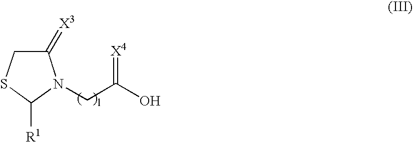

where l, R¹, X³, and X⁴ are defined as above, with either (i) a suitable primary or secondary amine according to the formula (HNR²R³) where R² and R³ are defined as above, or (ii) ammonia in the presence of an R²—H containing compound, under conditions effective to form the compound according to formula (I).

A sixth aspect of the present invention relates to a method of making a compound according to formula (II) that includes the steps of: reacting an intermediate according to formula (IV),

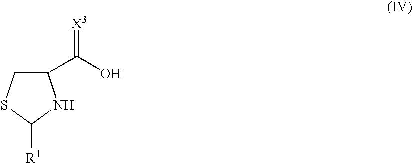

where R¹ and X³ are defined as above, with a primary or secondary amine according to the formula (HNR²R³) where R² and R³ are defined as above, under conditions effective to form the compound according to formula (II).

A seventh aspect of the present invention relates to intermediate compounds according to formula (III) and formula (IV).

An eighth aspect of the present invention relates to the use of the carboxylic acid intermediates of formula (III) or (IV) in the formation of a polymeric conjugate that includes at least one reactive amine group. Preferably, the polymeric conjugate constitutes a polyamine in accordance with the definitions of R² and R¹⁰ above.

A ninth aspect of the present invention relates to polymeric conjugates of serine amide alcohols, phosphates, thiophosphates, or phosphonates according to formula (V)

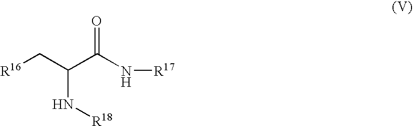

where R¹⁶ is a hydroxyl, phosphate, thiophosphate, or phosphonate; and R¹⁷ is a polymeric conjugated as described herein.

A tenth aspect of the present invention relates to a compound having a formula

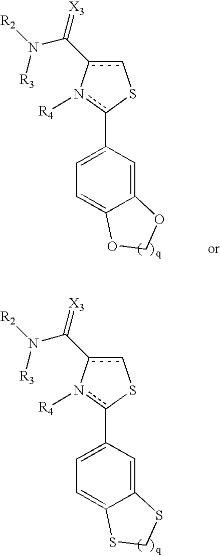

wherein q is 1 or 2, and X³, R², R³, and R⁴ are as defined above.

An eleventh aspect of the present invention relates to a method of destroying a cancer cell. This method involves providing a compound according to the tenth aspect of the present invention and contacting the cancer cell with the compound under conditions effective to kill the cancer cell.

A twelfth aspect of the present invention relates to a method of treating cancer. This method involves providing a compound according to the tenth aspect of the present invention and administering the compound to a patient having cancer, where the administering is effective to kill cancer cells and thereby treat the cancer.

A thirteenth aspect of the present invention relates to a method of making a compound according to the tenth aspect of the present invention. This method involves providing a first intermediate compound having a formula

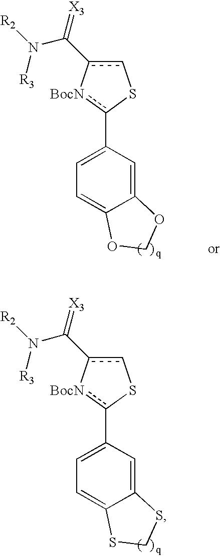

wherein Boc is a protective group and q, X₃, R₂, and R₃ are as defined above, and converting the first intermediate compound to the compound.

The present invention affords a significant improvement over previously identified cancer therapeutics that are known to be useful for the inhibition of prostate cancer cell growth. In a previous report, it was shown that cytotoxic compounds were obtained by replacing the glycerol backbone in LPA with serine amide in five prostate cancer cell lines (Gududuru et al., “Synthesis and Biological Evaluation of Novel Cytotoxic Phospholipids for Prostate Cancer,” Bioorg. Med. Chem. Lett. 14:4919-4923 (2004), which is hereby incorporated by reference in its entirety). The most potent compounds reported in Gududuru et al. (cited above) were non-selective and potently killed both prostate cancer and control cell lines. The present invention affords compounds that possess similar or even improved potency, but more importantly, improved selectivity, particularly with respect to prostate cancer cell lines. Compounds of the present invention are shown to be effective against prostate cancer cells, ovarian cancer cells, and skin cancer (melanoma) cells.

### BRIEF DESCRIPTION OF THE DRAWINGS

FIG. 1 illustrates one approach (scheme 1) for the synthesis of thiazolidine carboxylic acid amides. The thiazolidine carboxylic acid intermediate (2a-v) is formed upon reacting L-cysteine with various aldehydes under reported conditions (Seki et al., “A Novel Synthesis of (+)-Biotin from L-Cysteine,” J. Org. Chem. 67:5527-5536 (2002), which is hereby incorporated by reference in its entirety). The intermediate carboxylic acid is reacted with an amine to form the corresponding amide (3-27).

FIG. 2 illustrates one approach (scheme 2) for the synthesis of N-acyl and N-sulfonyl derivatives of the thiazolidine carboxylic acid amides. The N-acyl and N-sulfonyl derivatives (compounds 28 and 29) were synthesized from compound 5 by standard procedures.

FIG. 3 illustrates one approach (scheme 3) for the synthesis of thiazole carboxylic acid amides. The thiazolidine carboxylic acid methyl ester was converted to the thiazole carboxylic acid methyl ester following a reported procedure (Badr et al., “Synthesis of Oxazolidines, Thiazolidines, and 5,6,7,8-Tetrahydro-1H, 3H-pyrrolo[1,2-c]Oxazole (or Thiazole)-1,3-diones from β-Hydroxy- or β-Mercapto-α-amino Acid Esters,” Bull. Chem. Soc. Jpn. 54:1844-1847 (1981), which is hereby incorporated by reference in its entirety), and then converted to the alkylamide.

FIG. 4 illustrates one approach (scheme 4) for the synthesis of 4-thiazolidinone carboxylic acids, and their conversion to corresponding amides by reaction with primary or secondary amines (HNR²R³). As shown in this reaction scheme, different starting materials (where l differs) can be used to prepare various compounds of the invention.

FIG. 5 illustrates a second approach (scheme 5) for the synthesis of 4-thiazolidinone carboxylic acids, and their conversion to corresponding amides by reaction with R²—CNO.

FIG. 6 illustrates three approaches for modifying the core structure of the thiazolidinone compounds of the present invention (scheme 6) to afford ring-bound sulfone or sulfoxide groups (steps a and b, respectively), as well as the complete reduction of carbonyl groups (step c).

FIG. 7 illustrates a process for the synthesis of polyamine conjugates of thiazolidinone amides (scheme 7).

FIG. 8A illustrates a process for the synthesis of polyamine reactants and carboxylic acid intermediates (scheme 8). FIG. 8B illustrates a process for the synthesis of polyamine derivatives of serine alcohols, serine amides, and 2-arylthiazolidine-4-carboxylic acid amides (scheme 9).

FIG. 9 illustrates a process for the general synthesis of 2-aryl-thiazolidine-4-carboxylic acid amides (scheme 10).

FIG. 10 illustrates a synthetic scheme where L-cysteine and appropriate benzonitriles were dissolved in a 1:1 (v/v) mixture of phosphate buffer (pH 6.4) and methanol and stirred at 50° C. to give cyclized 2-aryl-4,5-dihydro-thiazole-4-carboxylic acid, which was reacted with tetradecylamine using EDC/HOBt to give corresponding compounds 328 and 329 (scheme 11).

FIG. 11 illustrates a synthetic scheme where derivatives 326-327 with a 4-amino-phenyl group were synthesized by deacetylation of compounds 314 and 317, which was accomplished by acid hydrolysis in methanol (scheme 12).

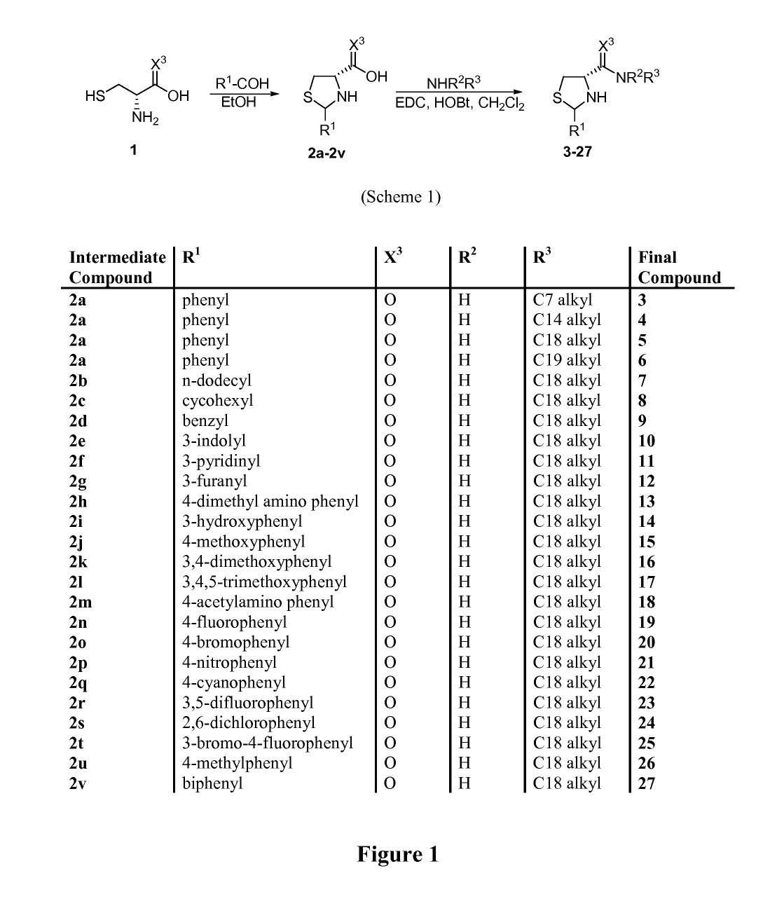

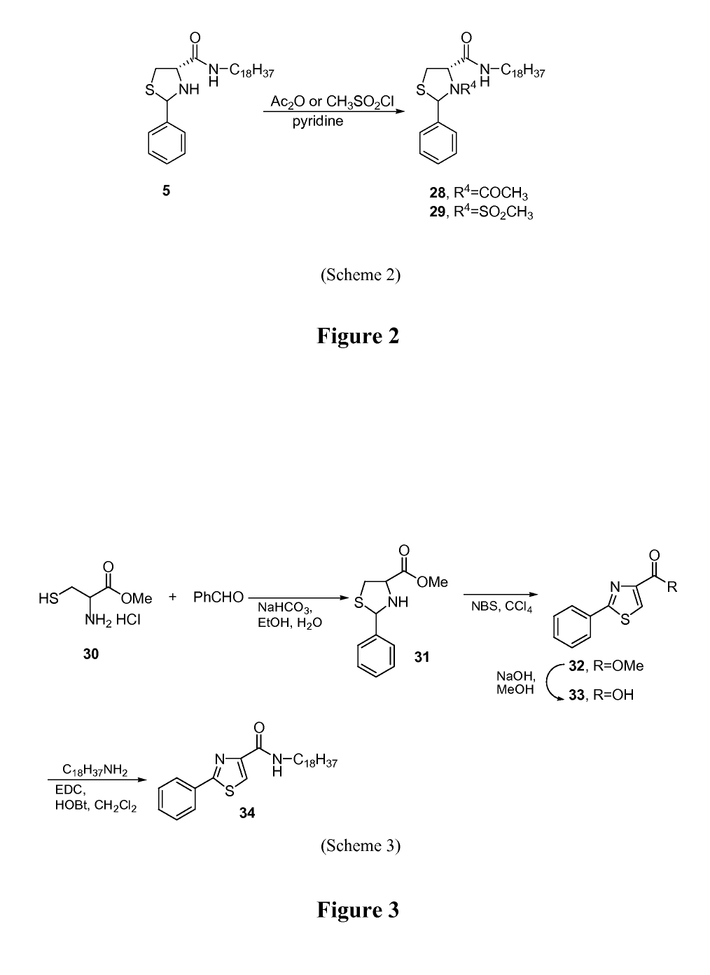

### DETAILED DESCRIPTION OF THE INVENTION

One aspect of the invention relates to compounds according to formulae (I) and (II) below

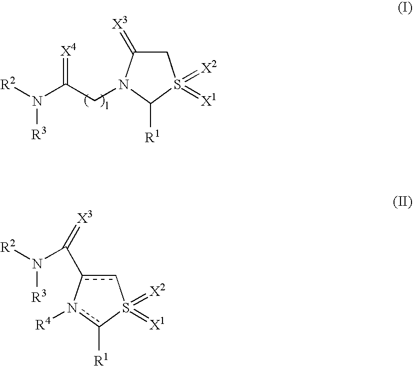

wherein

X¹ and X² are each optional, and each can be oxygen;

X³ and X⁴ are each optional, and each can be oxygen or sulfur;

l is an integer from 1 to 12;

R¹ is selected from the group of saturated or unsaturated cyclic hydrocarbons, saturated or unsaturated N-heterocycles, saturated or unsaturated O-heterocycles, saturated or unsaturated S-heterocycles, saturated or unsaturated mixed heterocycles, aliphatic or non-aliphatic straight- or branched-chain C1 to C30 hydrocarbons, or

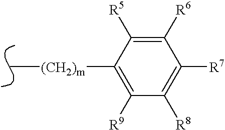

or —(CH₂)ₘ—Y¹ where m is an integer from 0 to 10 and Y¹ is a saturated or unsaturated cyclic hydrocarbon, saturated or unsaturated N-heterocycle, saturated or unsaturated O-heterocycle, saturated or unsaturated S-heterocycle, or saturated or unsaturated mixed heterocycle;

R² is hydrogen, alkoxy, an aliphatic or non-aliphatic straight- or branched-chain C1 to C30 hydrocarbon, R¹⁰—N(Z)-hydrocarbon- or R¹⁰-hydrocarbon- where the hydrocarbon group is an aliphatic or non-aliphatic straight- or branched-chain C1 to C30 hydrocarbon, a saturated or unsaturated cyclic hydrocarbons, a saturated or unsaturated N-heterocycle, a saturated or unsaturated O-heterocycle, a saturated or unsaturated S-heterocycle, a saturated or unsaturated mixed heterocycle,

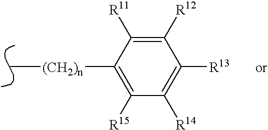

or —(CH₂)ₙ—Y² where n is an integer from 0 to 10 and Y² is a saturated or unsaturated cyclic hydrocarbon, saturated or unsaturated N-heterocycle, saturated or unsaturated O-heterocycle, saturated or unsaturated S-heterocycle, or saturated or unsaturated mixed heterocycle;

R³ is hydrogen, alkoxy, or an aliphatic or non-aliphatic straight- or branched-chain C1 to C10 hydrocarbon;

R⁴ is optional, or can be hydrogen, an aliphatic or non-aliphatic straight- or branched-chain C1 to C10 hydrocarbon, acyl, acetyl, or mesyl;

R⁵, R⁶, R⁷, R⁸, R⁹, R¹⁰, R¹¹, R¹², R¹³, R¹⁴, and R¹⁵ are independently selected from the group of hydrogen, hydroxyl, an aliphatic or non-aliphatic straight- or branched-chain C1 to C10 hydrocarbon, alkoxy, aryloxy, nitro, cyano, chloro, fluoro, bromo, iodo, haloalkyl, dihaloalkyl, trihaloalkyl, amino, alkylamino, dialkylamino, acylamino, arylamino, amido, alkylamido, dialkylamido, arylamido, aryl, C5 to C7 cycloalkyl, and arylalkyl; or any one or more combinations of R⁵ and R⁶, R⁶ and R⁷, R⁷ and R⁸, or R⁸ and R⁹ form a dioxolyl ring (—O—CH₂—O—) or a dioxanyl ring (—O—CH₂—CH₂—O—), or a dithiolanyl ring (—S—CH₂—S—) or a dithianyl ring (—S—CH₂—CH₂—S—) ring;

R¹⁰ is H(Z)N—, H(Z)N-hydrocarbon-, H(Z)N-hydrocarbon-N(Z)-hydrocarbon-, H(Z)N-hydrocarbon-N(Z)-hydrocarbon-N(Z)-hydrocarbon-, H(Z)N-hydrocarbon-O-hydrocarbon-, H(Z)N-hydrocarbon-O-hydrocarbon-N(Z)-hydrocarbon-, hydrocarbon-O-hydrocarbon-, hydrocarbon-N(Z)-hydrocarbon-, H(Z)N-hydrocarbon-carbonyl-hydrocarbon-, hydrocarbon-carbonyl-hydrocarbon-, H(Z)N-phenyl-, H(Z)N-phenylalkyl-, H(Z)N-phenylalkyl-N(Z)-hydrocarbon-, H(Z)N-phenylalkyl-N(Z)-hydrocarbon-N(Z)-hydrocarbon-, H(Z)N-phenylalkyl-O-hydrocarbon-, H(Z)N-phenylalkyl-O-hydrocarbon-N(Z)-hydrocarbon-, phenylalkyl-O-hydrocarbon-, phenylalkyl-N(Z)-hydrocarbon-, H(Z)N-phenylalkyl-carbonyl-hydrocarbon-, or phenylalkyl-carbonyl-hydrocarbon-, wherein each hydrocarbon is independently an aliphatic or non-aliphatic straight- or branched-chain C1 to C10 group, and wherein each alkyl is a C1 to C10 alkyl; and

Z is independently hydrogen or t-butoxycarbonyl.

As used herein, “aliphatic or non-aliphatic straight- or branched-chain hydrocarbon” refers to both alkylene groups that contain a single carbon and up to a defined upper limit, as well as alkenyl groups and alkynyl groups that contain two carbons up to the upper limit, whether the carbons are present in a single chain or a branched chain. Unless specifically identified, a hydrocarbon can include up to about 30 carbons, or up to about 20 hydrocarbons, or up to about 10 hydrocarbons.

As used herein, the term “alkyl” can be any straight- or branched-chain alkyl group containing up to about 30 carbons unless otherwise specified. The alkyl group can be a sole constituent or it can be a component of a larger constituent, such as in an alkoxy, arylalkyl, alkylamino, etc.

As used herein, “saturated or unsaturated cyclic hydrocarbons” can be any such cyclic hydrocarbon, including but not limited to phenyl, biphenyl, triphenyl, naphthyl, cycloalkyl, cycloalkenyl, cyclodienyl, etc.; “saturated or unsaturated N-heterocycles” can be any such N-containing heterocycle, including but not limited to aza- and diaza-cycloalkyls such as aziridinyl, azetidinyl, diazatidinyl, pyrrolidinyl, piperidinyl, piperazinyl, and azocanyl, pyrrolyl, pyrazolyl, imidazolyl, pyridinyl, pyrimidinyl, pyrazinyl, pyridazinyl, triazinyl, tetrazinyl, pyrrolizinyl, indolyl, quinolinyl, isoquinolinyl, benzimidazolyl, indazolyl, quinolizinyl, cinnolinyl, quinalolinyl, phthalazinyl, naphthyridinyl, quinoxalinyl, etc.; “saturated or unsaturated O-heterocycles” can be any such O-containing heterocycle including but not limited to oxiranyl, oxetanyl, tetrahydrofuranyl, tetrahydropyranyl, dioxanyl, furanyl, pyrylium, benzofuranyl, etc.; “saturated or unsaturated S-heterocycles” can be any such S-containing heterocycle, including but not limited to thiranyl, thietanyl, tetrahydrothiophenyl, dithiolanyl, tetrahydrothiopyranyl, thiophenyl, thiepinyl, thianaphthenyl, etc.; “saturated or unsaturated mixed heterocycles” can be any heterocycle containing two or more S—, N—, or O-heteroatoms, including but not limited to oxathiolanyl, morpholinyl, thioxanyl, thiazolyl, isothiazolyl, thiadiazolyl, oxazolyl, isoxazolyl, oxadiaziolyl, etc.

Preferred R¹ groups include benzyl, furanyl, indolyl, pyridinyl, phenyl, or substituted phenyl (with R⁵-R⁹ as defined above).

Preferred R² groups include methoxy, saturated and unsaturated aliphatic or non-aliphatic straight- or branched-chain C1 to C30 hydrocarbons, phenyl, phenylalkyls, substituted phenyls and substituted phenylalkyls with R¹¹-R¹⁵ groups as defined above. Preferred aliphatic or non-aliphatic straight- or branched-chain hydrocarbons are C8 to C24 saturated or monounsaturated hydrocarbons, including C10 to C20 alkyls or alkenyls, more preferably C14 to C18 alkyls or alkenyls.

Preferred R³ groups include hydrogen, methoxy, and C1 to C10 alkyls.

Preferred R⁴ groups include hydrogen, acyl, acetyl, and mesyl.

Preferred R¹⁰ groups are polyamines.

The integer l is preferably from 1 to 10, more preferably 1 to 8, 1 to 6, or 1 to 4. The integer m is preferably from 0 to 8, 0 to 6, 0 to 4, or 0 to 2. The integer n is preferably from 0 to 8, 0 to 6, 0 to 4, or 0 to 2.

Exemplary compounds according to formula (I) include, without limitation: 2-(4-oxo-2-phenylthiazolidin-3-yl)acetamide (compound 65), N-decyl-2-(4-oxo-2-phenylthiazolidin-3-yl)acetamide (compound 66), N-tetradecyl-2-(4-oxo-2-phenylthiazolidin-3-yl)acetamide (compound 67), N-octadecyl-2-(4-oxo-2-phenylthiazolidin-3-yl)acetamide (compound 68), N-octadecyl-2-(4-oxo-2-biphenylthiazolidin-3-yl)acetamide (compound 69), 2-(2-(1-(dimethylamino)naphthalen-4-yl)-4-oxothiazolidin-3-yl)-N-octadecylacetamide (compound 70), 2-(2-(4-methoxyphenyl)-4-oxothiazolidin-3-yl)-N-octadecylacetamide (compound 71), 2-(2-(2,6-dichlorophenyl)-4-oxothiazolidin-3-yl)-N-octadecylacetamide (compound 72), N-octadecyl-2-(4-oxo-2-phenyl-1-sulfoxide-thiazolidin-3-yl)acetamide (compound 80), N-octadecyl-2-(4-oxo-2-phenyl-1-sulfonyl-thiazolidin-3-yl)acetamide (compound 81), N-(3,5-difluorophenyl)-2-(4-oxo-2-phenylthiazolidin-3-yl)acetamide (compound 73), N-(3,5-difluorophenyl)-2-(4-oxo-2-phenylthiazolidin-3-yl)ethanethioamide, N-(3,5-bis(trifluoromethyl)phenyl)-2-(4-oxo-2-phenylthiazolidin-3-yl)acetamide (compound 74), N-(3,5-dichlorophenyl)-2-(4-oxo-2-phenylthiazolidin-3-yl)acetamide (compound 75), N-(2,4-dimethoxyphenyl)-2-(4-oxo-2-phenylthiazolidin-3-yl)acetamide (compound 76), N-(naphthalen-1-yl)-2-(4-oxo-2-phenylthiazolidin-3-yl)acetamide (compound 77), 3-(2-(octadecylamino)ethyl)-2-phenylthiazolidin-4-one (compound 79), N-(2-(2-phenylthiazolidin-3-yl)ethyl)octadecan-1-amine, and salts thereof.

Preferred compounds according to formula (I) include compounds 68, 71, 80, and 81.

Exemplary compounds according to formula (II) include, without limitation: (4R)-2-(4-methoxyphenyl)-N-octadecylthiazolidine-4-carboxamide (compound 15); (4R)-2-(4-ethoxyphenyl)-N-octadecylthiazolidine-4-carboxamide; N-octadecyl-2-phenylthiazole-4-carboxamide (compound 34); (4R)-2-(3,5-difluorophenyl)-N-octadecylthiazolidine-4-carboxamide (compound 23); (4R)-2-(4-cyanophenyl)-N-octadecylthiazolidine-4-carboxamide (compound 22); (4R)—N-octadecyl-N-mesyl-2-phenylthiazolidine-4-carboxamide (compound 29); (4R)—N-octadecyl-N-acetyl-2-phenylthiazolidine-4-carboxamide (compound 28); (4R)—N-heptyl-2-phenylthiazolidine-4-carboxamide (compound 3); (4R)—N-octadecyl-2-phenylthiazolidine-4-carboxamide (compound 5, R-isomer); (4S)—N-octadecyl-2-phenylthiazolidine-4-carboxamide (compound 5, S-isomer); (4R)—N-tetradecyl-2-phenylthiazolidine-4-carboxamide hydrochloride (compound 4, R-isomer); (4S)—N-tetradecyl-2-phenylthiazolidine-4-carboxamide hydrochloride (compound 4, S-isomer); (4R)—N-octadecyl-2-biphenylthiazolidine-4-carboxamide (compound 27); (4R)-2-dodecyl-N-octadecylthiazolidine-4-carboxamide (compound 7); (4R)—N-octadecyl-2-(pyridin-3-yl)thiazolidine-4-carboxamide (compound 11); 2-(furan-3-yl)-N-octadecylthiazolidine-4-carboxamide (compound 12); (4R)—N-nonadecyl-2-phenylthiazolidine-4-carboxamide (compound 6); (4R)-2-(4-hydroxyphenyl)-N-octadecylthiazolidine-4-carboxamide; 2-(3-hydroxyphenyl)-N-octadecylthiazolidine-4-carboxamide (compound 14); (4R)-2-(2,4,6-trimethoxyphenyl)-N-octadecylthiazolidine-4-carboxamide; 2-(3,4-dimethoxyphenyl)-N-octadecylthiazolidine-4-carboxamide (compound 16); 2-(3,4,5-trimethoxyphenyl)-N-octadecylthiazolidine-4-carboxamide (compound 17); (4R)-2-(4-acetamidophenyl)-N-octadecylthiazolidine-4-carboxamide (compound 18, R-isomer); (4S)-2-(4-acetamidophenyl)-N-octadecylthiazolidine-4-carboxamide (compound 18, S-isomer); (4R)-2-(4-fluorophenyl)-N-octadecylthiazolidine-4-carboxamide (compound 19); (4R)-2-(2,6-dichlorophenyl)-N-octadecylthiazolidine-4-carboxamide (compound 24); (4R)-2-(4-bromophenyl)-N-octadecylthiazolidine-4-carboxamide (compound 20); (4R)—N-octadecyl-2-p-tolylthiazolidine-4-carboxamide (compound 26); (4R)-2-cyclohexyl-N-octadecylthiazolidine-4-carboxamide (compound 8, R-isomer); (4S)-2-cyclohexyl-N-octadecylthiazolidine-4-carboxamide (compound 8, S-isomer) 2-(4-nitrophenyl)-N-octadecylthiazolidine-4-carboxamide (compound 21); (4R)-2-(4-(dimethylamino)phenyl)-N-octadecylthiazolidine-4-carboxamide (compound 13); (4R)-2-(1H-indol-3-yl)-N-octadecylthiazolidine-4-carboxamide (compound 10); (4R)-2-benzyl-N-octadecylthiazolidine-4-carboxamide (compound 9); (4R)-2-(3-bromo-4-fluorophenyl)-N-octadecylthiazolidine-4-carboxamide (compound 25); (4R)-2-(3,4,5-trimethoxyphenyl)-N,N-dioctylthiazolidine-4-carboxamide; (4R)-2-(4-methoxyphenyl)-N-tetradecylthiazolidine-4-carboxamide; (4S)-2-(4-methoxyphenyl)-N-tetradecylthiazolidine-4-carboxamide; (4R)-2-(2,4,6-trimethoxyphenyl)-N-tetradecylthiazolidine-4-carboxamide; (4S)-2-(2,4,6-trimethoxyphenyl)-N-tetradecylthiazolidine-4-carboxamide; (4S)-2-(3,4-dimethoxyphenyl)-N-tetradecylthiazolidine-4-carboxamide; (4S)-2-(4-acetamidophenyl)-N-tetradecylthiazolidine-4-carboxamide; (4R)-2-(3,4,5-trimethoxyphenyl)-N-octylthiazolidine-4-carboxamide (compound 301); (4R)—N-decyl-2-(3,4,5-trimethoxyphenyl)thiazolidine-4-carboxamide (compound 302); (4R)—N-dodecyl-2-(3,4,5-trimethoxyphenyl)thiazolidine-4-carboxamide (compound 303); (4R)-2-(3,4,5-trimethoxyphenyl)-N-tetradecylthiazolidine-4-carboxamide (compound 304); (4R)—N-hexadecyl-2-(3,4,5-trimethoxyphenyl)thiazolidine-4-carboxamide (compound 305); (4R)—N-decyl-2-(3,4-dimethoxyphenyl)thiazolidine-4-carboxamide (compound 306); (4R)—N-dodecyl-2-(3,4-dimethoxyphenyl)thiazolidine-4-carboxamide (compound 307); (4R)-2-(3,4-dimethoxyphenyl)-N-tetradecylthiazolidine-4-carboxamide (compound 308); (4R)—N-hexadecyl-2-(3,4-dimethoxyphenyl)thiazolidine-4-carboxamide (compound 309); (4R)-2-(benzo[d][1,3]dioxol-5-yl)-N-decylthiazolidine-4-carboxamide (compound 310); (4R)-2-(benzo[d][1,3]dioxol-5-yl)-N-dodecylthiazolidine-4-carboxamide (compound 311); (4R)-2-(benzo[d][1,3]dioxol-5-yl)-N-tetradecylthiazolidine-4-carboxamide (compound 312); (4R)-2-(benzo[d][1,3]dioxol-5-yl)-N-hexadecylthiazolidine-4-carboxamide (compound 313); (4R)-2-(2-acetamidophenyl)-N-dodecylthiazolidine-4-carboxamide (compound 314a); (4R)-2-(3-acetamidophenyl)-N-dodecylthiazolidine-4-carboxamide (compound 314b); (4R)-2-(4-acetamidophenyl)-N-dodecylthiazolidine-4-carboxamide (compound 314c); (4R)-2-(2-acetamidophenyl)-N-tetradecylthiazolidine-4-carboxamide (compound 315a); (4R)-2-(3-acetamidophenyl)-N-tetradecylthiazolidine-4-carboxamide (compound 315b); (4R)-2-(4-acetamidophenyl)-N-tetradecylthiazolidine-4-carboxamide (compound 315c); (4R)-2-(2-acetamidophenyl)-N-pentadecylthiazolidine-4-carboxamide (compound 316a); (4R)-2-(3-acetamidophenyl)-N-pentadecylthiazolidine-4-carboxamide (compound 316b); (4R)-2-(4-acetamidophenyl)-N-pentadecylthiazolidine-4-carboxamide (compound 316c); (4R)-2-(2-acetamidophenyl)-N-hexadecylthiazolidine-4-carboxamide (compound 317a); (4R)-2-(3-acetamidophenyl)-N-hexadecylthiazolidine-4-carboxamide (compound 317b); (4R)-2-(4-acetamidophenyl)-N-hexadecylthiazolidine-4-carboxamide (compound 317c); (4R)-2-(2-acetamidophenyl)-N-heptadecylthiazolidine-4-carboxamide (compound 318a); (4R)-2-(3-acetamidophenyl)-N-heptadecylthiazolidine-4-carboxamide (compound 318b); (4R)-2-(4-acetamidophenyl)-N-heptadecylthiazolidine-4-carboxamide (compound 318c); (4R)-2-(2-acetamidophenyl)-N-octadecylthiazolidine-4-carboxamide (compound 319a); (4R)-2-(3-acetamidophenyl)-N-octadecylthiazolidine-4-carboxamide (compound 319b); (4R)-2-(2-acetamidophenyl)-N-((Z)-octadec-8-enyl)thiazolidine-4-carboxamide (compound 320Z); (4R)-2-(2-acetamidophenyl)-N-((E)-octadec-8-enyl)thiazolidine-4-carboxamide (compound 120E); (4R)-2-(3-acetamidophenyl)-N-((Z)-octadec-8-enyl)thiazolidine-4-carboxamide (compound 320Z); (4R)-2-(3-acetamidophenyl)-N-((E)-octadec-8-enyl)thiazolidine-4-carboxamide (compound 320E); (4R)-2-(4-acetamidophenyl)-N-((Z)-octadec-8-enyl)thiazolidine-4-carboxamide (compound 320Z); (4R)-2-(4-acetamidophenyl)-N-((E)-octadec-8-enyl)thiazolidine-4-carboxamide (compound 320E); (4R)-2-(5-acetamidophenyl)-N-((Z)-octadec-8-enyl)thiazolidine-4-carboxamide (compound 320Z); (4R)-2-(5-acetamidophenyl)-N-((E)-octadec-8-enyl)thiazolidine-4-carboxamide (compound 320E); (4R)-2-(6-acetamidophenyl)-N-((Z)-octadec-8-enyl)thiazolidine-4-carboxamide (compound 320Z); (4R)-2-(6-acetamidophenyl)-N-((E)-octadec-8-enyl)thiazolidine-4-carboxamide (compound 320E); (4R)-2-phenyl-N-tetradecylthiazolidine-4-carboxamide (compound 321); (4R)—N-hexadecyl-2-phenylthiazolidine-4-carboxamide (compound 322); (4R)—N-methoxy-N-methyl-2-phenylthiazolidine-4-carboxamide (compound 323); (4S)-2-(3,4,5-trimethoxyphenyl)-N-tetradecylthiazolidine-4-carboxamide (compound 324); (4S)-2-(2-acetamidophenyl)-N-hexadecylthiazolidine-4-carboxamide (compound 325a); (4S)-2-(3-acetamidophenyl)-N-hexadecylthiazolidine-4-carboxamide (compound 325b); (4S)-2-(4-acetamidophenyl)-N-hexadecylthiazolidine-4-carboxamide (compound 325c); (4R)-2-(2-aminophenyl)-N-dodecylthiazolidine-4-carboxamide (compound 326a); (4R)-2-(3-aminophenyl)-N-dodecylthiazolidine-4-carboxamide (compound 326b); (4R)-2-(4-aminophenyl)-N-dodecylthiazolidine-4-carboxamide (compound 326c); (4R)-2-(2-aminophenyl)-N-hexadecylthiazolidine-4-carboxamide (compound 327a); (4R)-2-(3-aminophenyl)-N-hexadecylthiazolidine-4-carboxamide (compound 327b); (4R)-2-(4-aminophenyl)-N-hexadecylthiazolidine-4-carboxamide (compound 327c); (R)-4,5-dihydro-2-phenyl-N-tetradecylthiazole-4-carboxamide (compound 328); (R)-4,5-dihydro-2-(3,4-dimethoxyphenyl)-N-tetradecylthiazole-4-carboxamide (compound 329); (4R)-2-(2-acetamidophenyl)-N-hexadecyl-3-methylthiazolidine-4-carboxamide (compound 330); and salts thereof.

Preferred compounds according to formula (II) include compounds 4 (R-isomer), 5 (R- and S-isomers), 13, 14, 16, 17, 18, 19, 25, 26, 317, 320Z, and 320E.

The compounds of the present invention and their intermediates can be synthesized using commercially available or readily synthesized reactants.

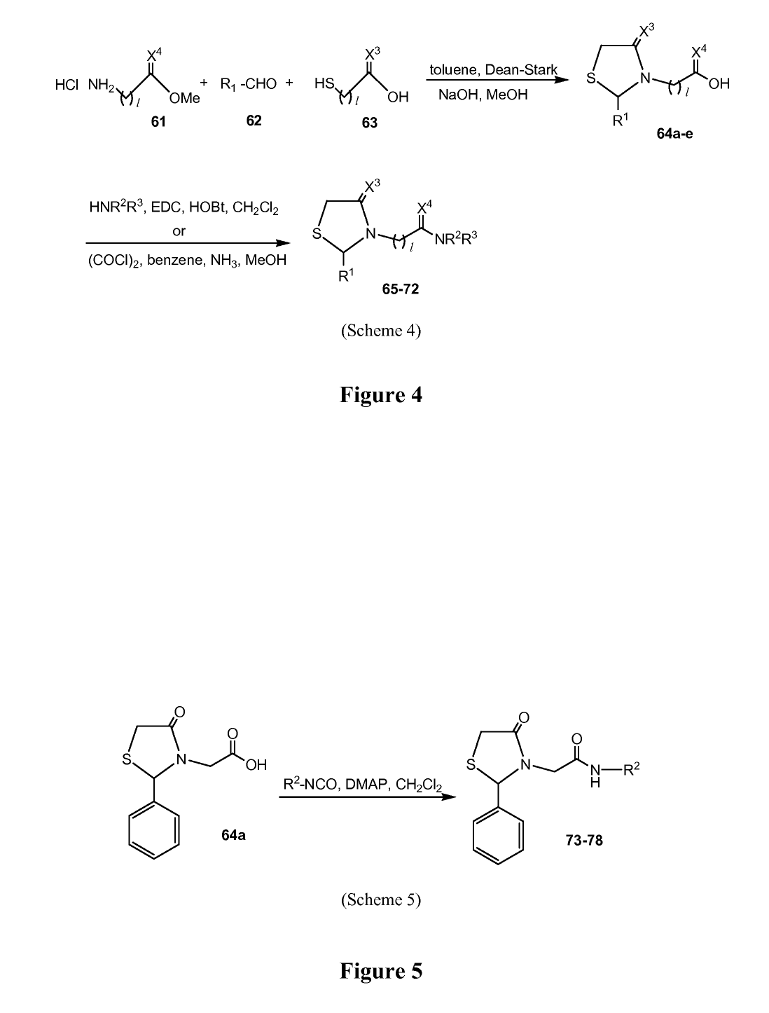

By way of example, the compounds according to formula (I) can be synthesized according to scheme 4 illustrated in FIG. 4. According to one approach, an intermediate acid according to formula (III)

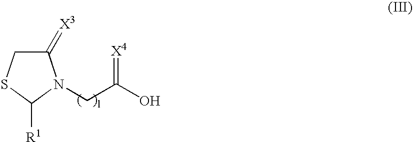

(where l, R¹, X³, and X⁴ are as defined above) is reacted with appropriate amines in the presence of EDC/HOBt under standard conditions. The intermediate acids can be prepared initially via condensing mercaptoacetic acid, glycine methyl ester, and aromatic aldehydes in a one-pot reaction, followed by basic hydrolysis of the ester (Holmes et al., “Strategies for Combinatorial Organic Synthesis: Solution and Polymer-Supported Synthesis of 4-Thiazolidinones and 4-Metathiazanones Derived from Amino Acids,” J. Org. Chem. 60:7328-7333 (1995), which is hereby incorporated by reference in its entirety). By substituting glycine methyl ester with analogs containing longer carbon backbones, it becomes possible to prepare compounds according to formula (III) and, ultimately, formula (I), with l being greater than 1 (i.e., containing an alkylene group that is longer than methylene). According to a second approach, the thiazolidinone amides of formula (I) can also be prepared by a simple and direct method (Schuemacher et al., “Condensation Between Isocyanates and Carboxylic Acids in the Presence of 4-Dimethylaminopyridine (DMAP), a Mild and Efficient Synthesis of Amides,” Synthesis 22:243-246 (2001), which is hereby incorporated by reference in its entirety), which involves reaction of the intermediate acid with desired isocyanates in the presence of a catalytic amount of DMAP (FIG. 5) (scheme 5).

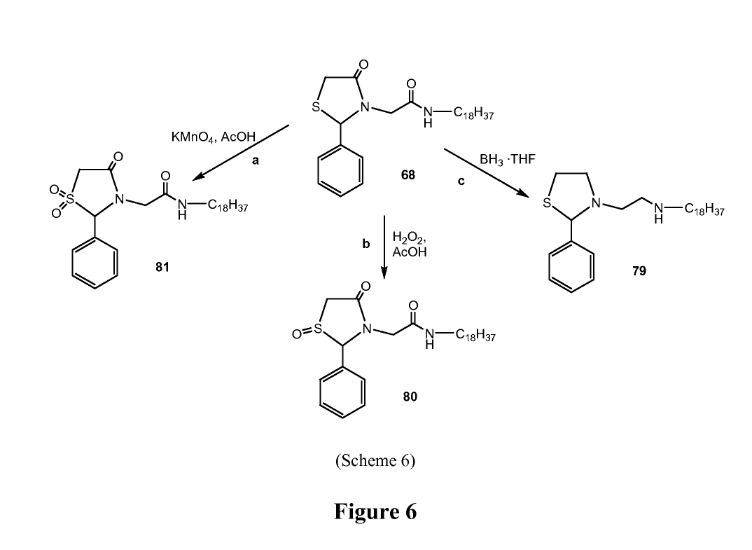

Further modification of the thiazolidinone compounds can be achieved by, e.g., exhaustive reduction of using BH₃.THF under reflux conditions to eliminate carbonyl or sulfoxide groups X³ and X⁴ (FIG. 6) (scheme 6c), as well as oxidation of a compound using H₂O₂ and KMnO₄ to afford sulfoxides or sulfones, respectively, as shown in scheme 6a and 6b.

Also by way of example, compounds according to formula (II) can be prepared by reacting an intermediate acid according to formula (IV),

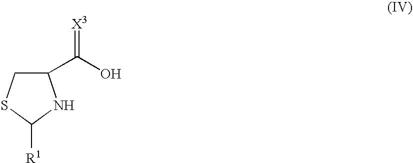

where compound (IV) can be either the R- or S-stereoisomer and R¹ and X³ are defined as above, with appropriate amines in the presence of EDC/HOBt under standard conditions. The intermediate acids can be prepared via reaction of L-cysteine with desired aldehydes under reported conditions (Seki et al., “A Novel Synthesis of (+)-Biotin from L-Cysteine,” J. Org. Chem. 67:5527-5536 (2002), which is hereby incorporated by reference in its entirety).

The compounds of the present invention can also be modified to contain a polymeric conjugate (i.e., as defined by the substituents R₂ and R₁₀). Suitable polymeric conjugates include, without limitation, poly(alkyl)amines, poly(alkoxy)amine, polyamines, etc. It is also well known that polyamine containing compounds exhibit a number of biological activities and have been utilized as chemotherapeutic agents. Exemplary conjugates include those containing the naturally occurring polyamines like putrescine, spermidine, and spermine, as well as synthetic polyamines.

A further aspect of the present invention relates to polymeric conjugates of a third class of compounds, polymeric conjugates of the serine amide alcohols and serine amide phosphates. These compounds are characterized by the structure according to formula (V)

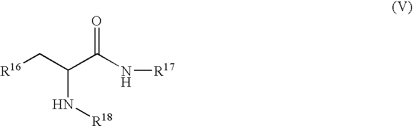

where

R¹⁶ is a hydroxyl group, phosphate group (H₂O₂P—O— or HO₂PO⁻—O—), thiophosphate group (H₂O₂PS— or HO₂PS⁻—O—), or phosphonate group (H₂O₂PO—CH₂— or HO₂PO⁻CH₂—);

R¹⁷ is defined above as R² contain an R¹⁰ substituent (i.e., R¹⁰—N(Z)-hydrocarbon- or R¹⁰-hydrocarbon-, where R¹⁰, Z, and hydrocarbon are defined above; and

R¹⁸ is defined as hydrogen, a straight or branched-chain C1 to C30 alkyl, a straight or branched-chain C2 to C30 alkenyl, an aromatic or heteroaromatic ring with or without mono-, di-, or tri-substitutions of the ring, an acyl including a C1 to C30 alkyl or an aromatic or heteroaromatic ring, an arylalkyl including straight or branched-chain C1 to C30 alkyl, an aryloxyalkyl including straight or branched-chain C1 to C30 alkyl,

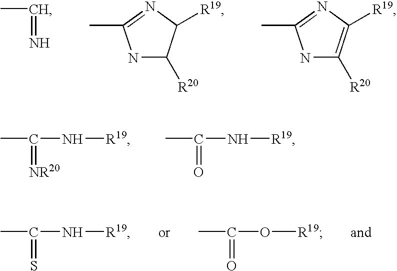

R¹⁹ and R²⁰ are independently hydrogen, a straight or branched-chain C1 to C30 alkyl, a straight or branched-chain C2 to C30 alkenyl, an aromatic or heteroaromatic ring with or without mono-, di-, or tri-substitutions of the ring, an acyl including a C1 to C30 alkyl or aromatic or heteroaromatic ring, an arylalkyl including straight or branched-chain C1 to C30 alkyl, or an aryloxyalkyl including straight or branched-chain C1 to C30 alkyl.

The synthesis of the serine amide alcohols, phosphates, phosphonates, and thiophosphates has been previously described in U.S. Pat. No. 6,875,757 to Miller et al.; U.S. patent application Ser. No. 10/963,085 to Miller et al.; and Gududuru et al., “Synthesis and Biological Evaluation of Novel Cytotoxic Phospholipids for Prostate Cancer,” Bioorg. Med. Chem. Lett. 14(19):4919-4923 (2004), each of which is hereby incorporated by reference in its entirety. The polymeric conjugates of these compounds can be prepared as described below and as demonstrated in the examples, infra.

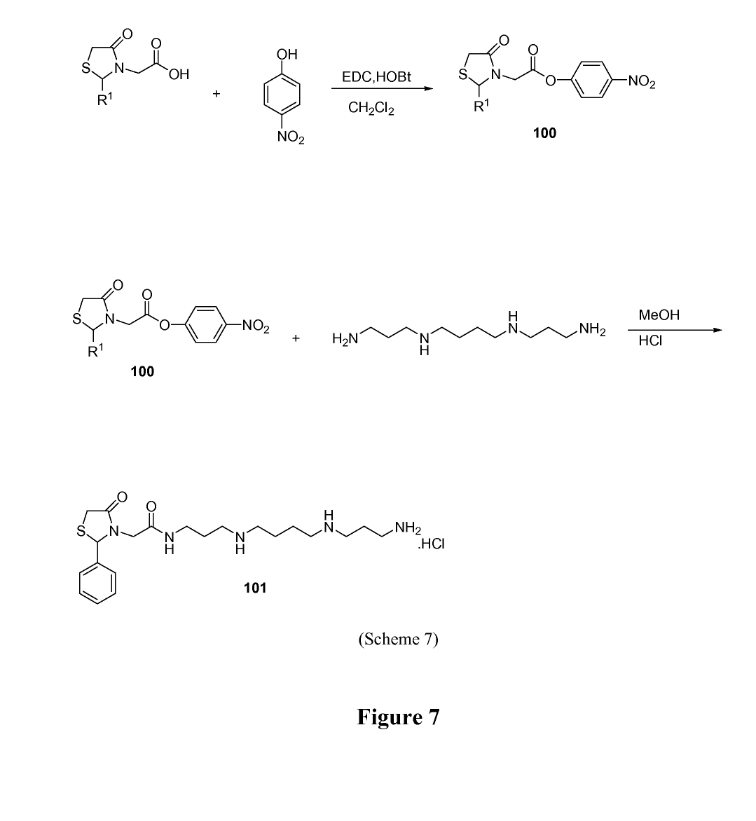

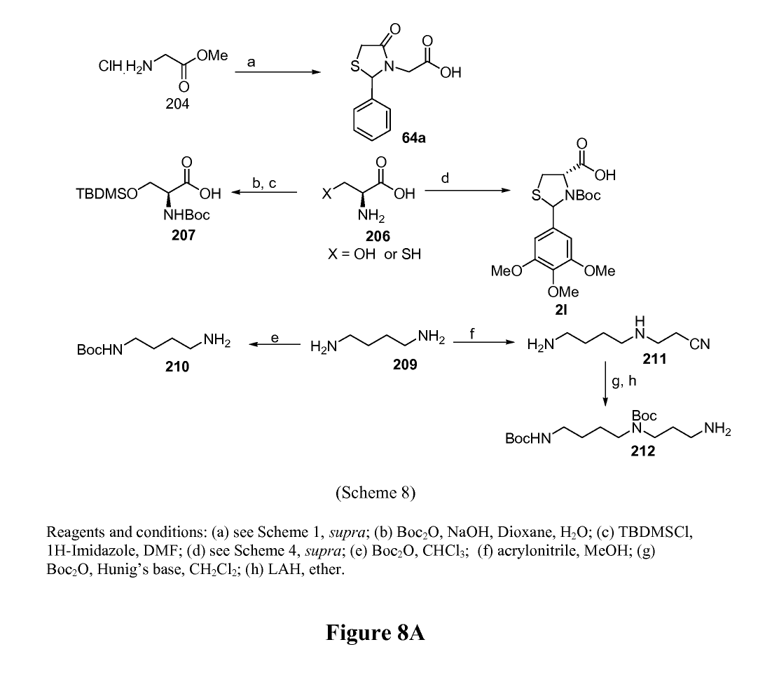

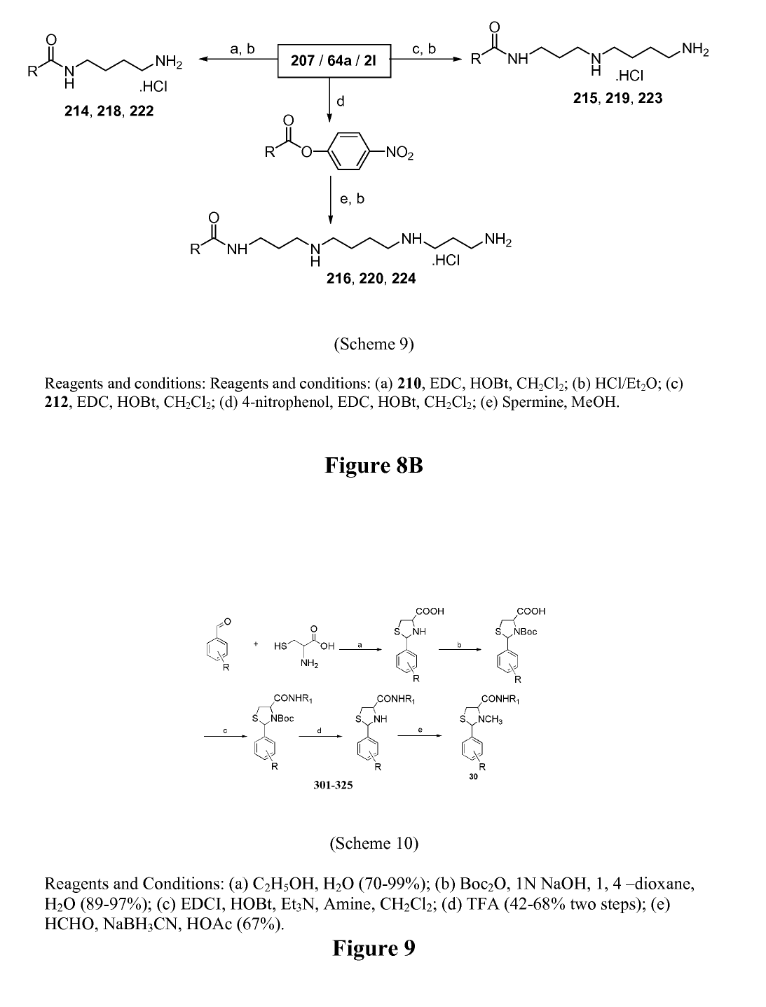

According to one approach, a compound of the present invention can be conjugated to a polyamine by reacting the intermediate acid or a nitrophenyl derivative thereof with a polyamine NH₂—R_{z} where R_{z} is any of the R²/R¹⁰ groups defined above. Exemplary synthesis schemes are illustrated in FIGS. 7-8.

The compounds can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

The compounds of the present invention can be present in the form of a racemic mixture, containing substantially equivalent amounts of stereoisomers. In another embodiment, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 95% pure, more preferably at least about 98% pure, most preferably at least about 99% pure.

Another aspect of the present invention relates to pharmaceutical compositions that contain one or more of the above-identified compounds of the present invention. Typically, the pharmaceutical composition of the present invention will include a compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. For example, application to mucous membranes can be achieved with an aerosol spray containing small particles of a compound of this invention in a spray or dry powder form.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In another embodiment, these compounds are tableted with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, these active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compounds of the present invention are particularly useful in the treatment or prevention of various forms of cancer, particularly prostate cancer, breast cancer, ovarian, and skin cancer (e.g., melanoma). It is believed that other forms of cancer will likewise be treatable or preventable upon administration of the compounds or compositions of the present invention to a patient. Preferred compounds of the present invention are selectively disruptive to cancer cells, causing ablation of cancer cells but not normal cells. Significantly, harm to normal cells is minimized because the cancer cells are susceptible to disruption at much lower concentrations of the compounds of the present invention.

Thus, a further aspect of the present invention relates to a method of destroying a cancerous cell that includes: providing a compound of the present invention and then contacting a cancerous cell with the compound under conditions effective to destroy the contacted cancerous cell. According to various embodiments of destroying the cancerous cells, the cells to be destroyed can be located either in vivo or ex vivo (i.e., in culture).

A still further aspect of the present invention relates to a method of treating or preventing a cancerous condition that includes: providing a compound of the present invention and then administering an effective amount of the compound to a patient in a manner effective to treat or prevent a cancerous condition.

According to one embodiment, the patient to be treated is characterized by the presence of a precancerous condition, and the administering of the compound is effective to prevent development of the precancerous condition into the cancerous condition. This can occur by destroying the precancerous cell prior to or concurrent with its further development into a cancerous state.

According to another embodiment, the patient to be treated is characterized by the presence of a cancerous condition, and the administering of the compound is effective either to cause regression of the cancerous condition or to inhibit growth of the cancerous condition. This preferably occurs by destroying cancer cells, regardless of their location in the patient body. That is, whether the cancer cells are located at a primary tumor site or whether the cancer cells have metastasized and created secondary tumors within the patient body.

As used herein, patient refers to any mammalian patient, including without limitation, humans and other primates, dogs, cats, horses, cows, sheep, pigs, rats, mice, and other rodents.

When administering the compounds of the present invention, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

When the compounds or pharmaceutical compositions of the present invention are administered to treat or prevent a cancerous condition, the pharmaceutical composition can also contain, or can be administered in conjunction with, other therapeutic agents or treatment regimen presently known or hereafter developed for the treatment of various types of cancer. Examples of other therapeutic agents or treatment regimen include, without limitation, radiation therapy, chemotherapy, surgical intervention, and combinations thereof.

Compositions within the scope of this invention include all compositions wherein the compound of the present invention is contained in an amount effective to achieve its intended purpose. While individual needs may vary, determination of optimal ranges of effective amounts of each component is within the skill of the art. Typical dosages comprise about 0.01 to about 100 mg/kg·body wt. The preferred dosages comprise about 0.1 to about 100 mg/kg·body wt. The most preferred dosages comprise about 1 to about 100 mg/kg·body wt. Treatment regimen for the administration of the compounds of the present invention can also be determined readily by those with ordinary skill in art. That is, the frequency of administration and size of the dose can be established by routine optimization, preferably while minimizing any side effects.

### EXAMPLES

The Examples set forth below are for illustrative purposes only and are not intended to limit, in any way, the scope of the present invention.

### Example 1

### Synthesis and Antiproliferative Activity of Thiazolidine Analogs for Melanoma

2-aryl-thiazolidine-4-carboxylic acid amides are shown in PCT Patent Application Nos. PCT/US2004/038662 and PCT/US2006/027763 (which are hereby incorporated by reference in their entirety) as a novel class of cytotoxic agents for prostate cancer. Screening these compounds with melanoma cell lines revealed that several of them have potent cytotoxicity and selectivity against melanoma (PCT Patent Application No. PCT/US2006/027763, which is hereby incorporated by reference in its entirety). To further improve the potency and selectivity, a new series of analogs was synthesized and tested in two melanoma cell lines and fibroblast cells (negative controls). Comparison of anticancer effects of these compounds with a standard chemotherapeutic agent, sorafenib, showed that they are very effective in killing melanoma cells with low micromolar to nanomolar cytotoxicity and provide a new lead for developing potential drugs for melanoma.

Recently, novel classes of lipid compounds have been synthesized and have shown strong activity toward prostate cancer cells (Gududuru et al., J. Med. Chem. 48:2584 (2005); Gududuru et al., Bioorg. Med. Chem. Lett. 15:4010 (2005)). These classes of compounds are unlikely to be DNA alkylating agents but are possibly interfering with lysophosphatidic acid (LPA) receptors in the cell membrane. Encouraged by the results with prostate cancer, it was decided to test a library of such compounds against metastatic melanoma in vitro. The initial results provided three classes of highly potent lead compounds for metastatic melanoma. The most potent lead compound has an IC50 value in the submicromole range with 10-fold selectivity against cancer cells. To further improve potency and selectivity, extensive synthesis and biological testing of additional compounds in this series was performed. The synthesis and in vitro cytotoxic activity of these new compounds against two human melanoma cell lines and fibroblast cells to determine their selectivity is reported below.

The general synthesis of 2-aryl-thiazolidine-4-carboxylic acid amides is shown in FIG. 9 (scheme 10).

L- or D-cysteine was reacted with appropriate benzaldehydes in ethanol and water at ambient temperature to give cyclized 2-aryl-thiazolidine-4-carboxylic acid, which was converted to the corresponding Boc derivatives. Reaction of Boc-protected carboxylic acids with different amines using EDC/HOBt gave corresponding amides, which were treated with TFA to form the target compounds 301-325 (Gududuru et al., J. Med. Chem. 48:2584 (2005), which is hereby incorporated by reference in its entirety). Reductive alkylation with formaldehyde and sodium cyanoborohydide of the amino group in compound 317 gave methylation derivative 330 (Borch et al., J. Org. Chem. 37:1673 (1972), which is hereby incorporated by reference in its entirety). Dimer 331 was obtained by intramolecular condensation of 2-aryl-thiazolidine-4-carboxylic acid with EDC/HOBt.

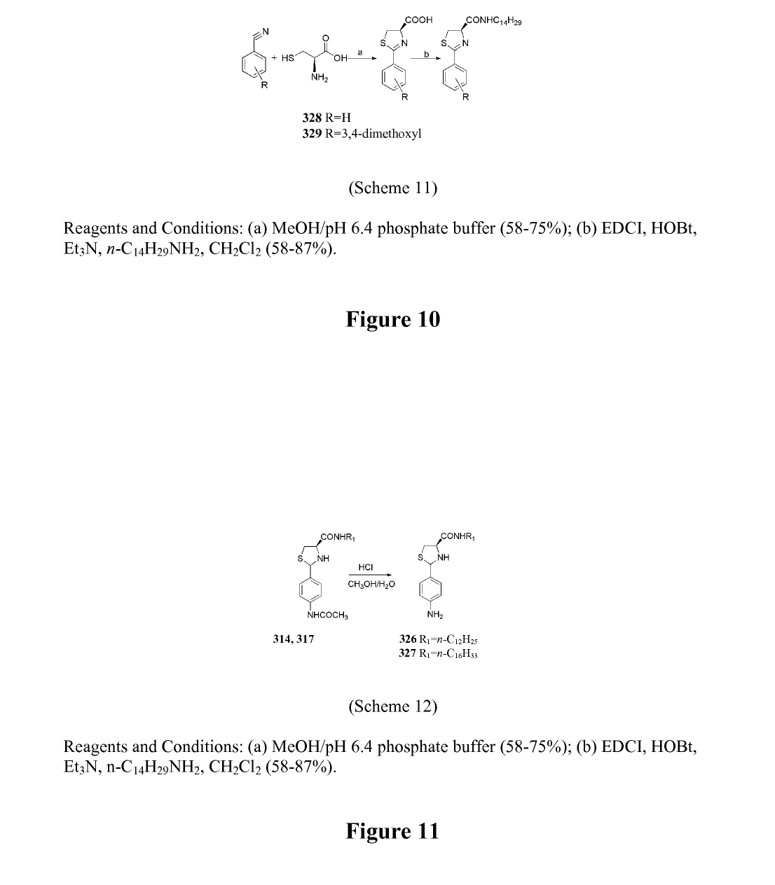

L-cysteine and appropriate benzonitriles were dissolved in a 1:1 (v/v) mixture of phosphate buffer (pH 6.4) and methanol and stirred at 50° C. to give cyclized 2-aryl-4,5-dihydro-thiazole-4-carboxylic acid, which was reacted with tetradecylamine using EDC/HOBt to give corresponding compounds 328 and 329 as shown in FIG. 10 (scheme 11) (Zamri et al., Tetrahedron 56:249 (2000), which is hereby incorporated by reference in its entirety).

Derivatives 326-327 with a 4-amino-phenyl group were synthesized by deacetylation of compounds 314 and 317, which was accomplished by acid hydrolysis in methanol as shown in FIG. 11 (scheme 12). Each compound was characterized with NMR, mass spectroscopy, and elemental analysis.

Cytotoxicity of these newly synthesized compounds was examined in two human melanoma cell lines (SK-MEL-188 and WM-164) and in a fibroblast cell line. Activity on fibroblast cells was used as a control to determine the selectivity of these compounds against melanoma. Standard sulforhodamine B (SRB) assay was used. Cells were exposed to a wide range of concentrations for 48 h in round-bottom 96-well plates. Cells were fixed with 10% trichloroacetic acid and washed five times with water. After cells were air-dried overnight and stained with SRB solution, total proteins were measured at 560 nm with a plate reader. IC₅₀ (i.e., concentration which inhibited cell growth by 50% of DMSO-treated controls) values were obtained by nonlinear regression analysis with GraphPad Prism (GraphPad Software, San Diego, Calif.).

The ability of thiazolidine derivatives to inhibit the growth of two melanoma cancer cell lines and fibroblast cells is summarized in Table 1. Sorafenib (Velcade) has been used extensively in clinical trials for melanoma, hence this compound and DTIC were selected as reference standards to assess the activity of the compounds. At this early stage, all compounds were used as a diastereomeric mixture if they contain chiral centers in order to select the most promising compounds for further development.

Examination of cytotoxic effects for a variety of substitutions on the phenyl ring revealed the chain-length dependence for these compounds (301-305, 306-309, 310-313, 314-319). Short chain length such as a C10 chain (for example, compound 302, 306, 310) displayed low potency for both cancer cells and fibroblast cells. As chain length increased, potency increased, as well as toxicity as measured on fibroblast cells except when the acetyl amino group was substituted on the phenyl ring (compound 314-317). Both C15 and C16 chains with this substitution displayed both high potency and high selectivity against cancer cells, with an IC₅₀ for melanoma cells as low as 600 nM (compound 317). Further chain increases, however, reduced potency and selectivity. At a chain length of C18 (compound 319), the IC₅₀ value was higher than 10 μM for all three cell lines. Interestingly, adding either a cis- or trans-double bond in the C18 side chain restored potency dramatically (compound 320Z and 320E), demonstrating that both length and composition of the side chain are critical for their activity. There is no significant difference in their activity between the cis- and trans-isomers.

TABLE 1
Antiproliferative activity of thiazolidine analogs and their comparison with that of sorafenib and DTIC
(ND: not detected). IC₅₀ values expressed with standard error.

| | | | | | IC₅₀ ± SEM (μM) | | |
|---|---|---|---|---|---|---|---|
| | | | | | SK-MEL- | | |
| Structure | Compd. | R | R₁ | R₂ | 188 | WM-164 | Fibroblast |
| 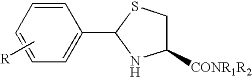 | 301 302 303 304 305 306 307 308 309 310311312313314315316317318319320Z 320E 321322323326327 | 3,4,5-trimethoxyl3,4,5-trimethoxyl3,4,5-trimethoxyl3,4,5-trimethoxyl3,4,5-trimethoxyl3,4-dimethoxyl3,4-dimethoxyl3,4-dimethoxyl3,4-dimethoxyl3,4-OCH₂O—3,4-OCH₂O—3,4-OCH₂O—3,4-OCH₂O—NHCOCH₃NHCOCH₃NHCOCH₃NHCOCH₃NHCOCH₃NHCOCH₃NHCOCH₃ NHCOCH₃ HHHNH₂NH₂ | n-C₈H₁₇ n-C₁₀H₂₁ n-C₁₂H₂₅ n-C₁₄H₂₉ n-C₁₆H₃₃ n-C₁₀H₂₁ n-C₁₂H₂₅ n-C₁₄H₂₉ n-C₁₆H₃₃ n-C₁₀H₂₁n-C₁₂H₂₅n-C₁₄H₂₉n-C₁₆H₃₃n-C₁₂H₂₅n-C₁₄H₂₉n-C₁₅H₃₁n-C₁₆H₃₃n-C₁₇H₃₅n-C₁₈H₃₇(Z)-Octadec-8-enyl(E)-Octadec-8-enyln-C₁₄H₂₉n-C₁₆H₃₃OCH₃n-C₁₂H₂₅n-C₁₆H₃₃ | H H H H H H H H H HHHHHHHHHHH H HHCH₃HH | 17.1 ± 0.6 14.5 ± 2.8 2.1 ± 0.4 2.0 ± 0.5 1.8 ± 0.2 11.9 ± 5.6 2.9 ± 0.9 1.5 ± 0.5 1.5 ± 0.6 6.6 ± 0.53.5 ± 0.11.6 ± 0.11.6 ± 0.12.4 ± 0.12.3 ± 01 1.6 ± 0.12.1 ± 0.28.5 ± 0.122.3 ± 2.8 1.4 ± 0.1 3.3 ± 0.4 1.9 ± 0.61.9 ± 0.1>1002.2 ± 0.12.3 ± 0.1 | 19.6 ± 0.9 2.1 ± 0.4 2.4 ± 0.4 1.6 ± 0.4 0.7 ± 0.1 6.1 ± 2.5 1.6 ± 0.5 0.8 ± 0.3 0.5 ± 0.2 4.5 ± 0.12.5 ± 0.11.0 ± 0.11.8 ± 0.11.2 ± 0.10.6 ± 0.11.0 ± 0.10.6 ± 0.12.4 ± 0.111.6 ± 0.6 1.0 ± 0.1 1.4 ± 0.2 0.6 ± 0.10.7 ± 0.1>1001.4 ± 0.11.4 ± 0.1 | 20.8 ± 10.4 6.7 ± 3.9 2.4 ± 1.2 2.6 ± 0.4 2.4 ± 0.4 6.3 ± 1.1 4.5 ± 1.9 2.8 ± 1.1 2.1 ± 0.8 8.2 ± 3.15.2 ± 1.24.2 ± 0.55.7 ± 1.83.5 ± 0.43.6 ± 0.814.3 ± 2.1 19.1 ± 7.7 35.8 ± 5.0 >60 10.6 ± 0.9 18.0 ± 3.5 2.8 ± 0.22.2 ± 0.2>1004.1 ± 0.57.4 ± 1.1 |
| 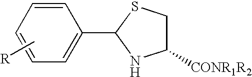 | 324 325 | 3,4,5-trimethoxylNHCOCH₃ | n-C₁₄H₂₉ n-C₁₆H₃₃ | H H | 2.6 ± 0.2 3.2 ± 0.2 | 1.1 ± 0.1 1.4 ± 0.1 | 4.1 ± 0.7 22.6 ± 3.0 |
| 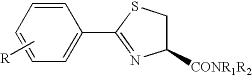 | 328329 | H3,4-dimethoxyl | n-C₁₄H₂₉n-C₁₄H₂₉ | HH | 42.1 ± 5.0 19.1 ± 1.8 | >50 42.9 ± 10.1 | >50 >50 |
| 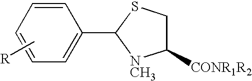 | 330 | NHCOCH₃ | n-C₁₆H₃₃ | H | 29.1 ± 1.0 | >100 | >100 |
| 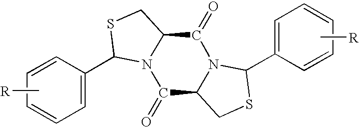 | 331 | 3,4,5-trimethoxyl | N/A | N/A | >50 | >50 | >50 |
| | DTIC | | | | >100 | >100 | ND |
| | Sora- | | | | 4.3 ± 0.2 | 4.7 ± 0.3 | >100 |
| | fenib | | | | | | |

Removing the acetyl amino group on the phenyl ring (compound 321 and 322) resulted in the loss of selectivity, although potency was similar to those with this substitution (compounds 315 and 317). Replacing the alkyl chain with a methoxyl group completely abolished potency (compound 323). Changing the chirality from an R to S configuration at the C4 position on the thiazolidine ring did not substantially affect either potency or selectivity (compound 304 vs 324 and compound 317 vs 325). Selectivity has a strong dependence on the substitutions in the phenyl ring. For example, with a C12 chain, potency is similar for all the substitutions studied (compounds 303, 307, 311, 314, and 326). However, selectivity improves dramatically when proper substitutions are present (compound 317 vs compounds 305, 309, 313, 322, and 327).

When the amino group in the thiazolidine ring is removed either by substitution (compound 330) or conjugation (compounds 328 and 329), the resulting compounds are largely inactive with IC₅₀ values above 20 μM. The intermediate compound in which the amino group is protected by a Boc group was also tested, and that compound is inactive also. Furthermore, when the aliphatic chain and the amino group was removed by synthesizing a dimer, an inactive compound (compound 331) was obtained. These results clearly demonstrate the importance of the amino group in the thiazolidine ring.

Not surprising, DTIC was inactive (IC50>100 μM) in the in vitro assay due to lack of bioactivation (Daidone et al., Farmaco 59:413 (2004), which is hereby incorporated by reference in its entirety). Recent clinical trials indicated that sorafenib has promising effect against melanoma, and it has very low toxicity (Eisen et al., Br. J. Cancer 95:581 (2006), which is hereby incorporated by reference in its entirety). The in vitro assay indicated that sorafenib was about 10 times less potent against melanoma cells than compound 317, but it had higher selectivity (less toxicity) as indicated by the ratio of its IC50 values for fibroblast cells over melanoma cells (larger than 25 for sorafenib vs 10˜20 for 317). The potency and selectivity of sorafenib provide an excellent standard to assess the activities of our compounds and its selectivity represents a goal for further optimizing lead structures.

In conclusion, novel analogs of thiazolidine compounds have been synthesized based on initial studies. When compared with existing anticancer drugs, these compounds were much more potent and moderately selective. Further optimization of the structure to improve selectivity is currently in progress. Once highly potent and selective compounds are identified, pure optical isomers will be separated by preparative HPLC for both in vitro and in vivo animal testing.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A compound having a formula 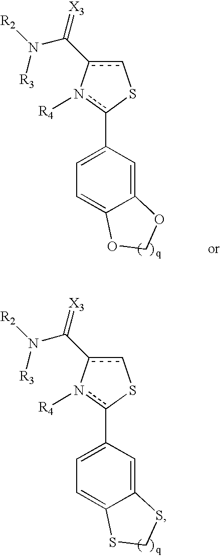
wherein
q is 1 or 2;
X³ is optional and can be oxygen or sulfur;
R² is hydrogen, alkoxy, an aliphatic or non-aliphatic straight- or branched-chain C1 to C30 hydrocarbon, R¹⁰—N(Z)-hydrocarbon- or R¹⁰-hydrocarbon-, where the hydrocarbon group is an aliphatic or non-aliphatic straight- or branched-chain C1 to C30 hydrocarbon, a saturated or unsaturated cyclic hydrocarbon, a saturated or unsaturated N-heterocycle, a saturated or unsaturated O-heterocycle, a saturated or unsaturated S-heterocycle, a saturated or unsaturated mixed heterocycle, 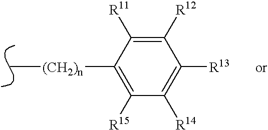
or —(CH₂)ₙ—Y² where n is an integer from 0 to 10 and Y² is a saturated or unsaturated cyclic hydrocarbon, saturated or unsaturated N-heterocycle, saturated or unsaturated O-heterocycle, saturated or unsaturated S-heterocycle, or saturated or unsaturated mixed heterocycle;
R³ is hydrogen, alkoxy, or an aliphatic or non-aliphatic straight- or branched-chain C1 to C10 hydrocarbon;
R⁴ is optional, or can be hydrogen, an aliphatic or non-aliphatic straight- or branched-chain C1 to C10 hydrocarbon, acyl, acetyl, or mesyl;
R¹¹, R¹², R¹³, R¹⁴, and R¹⁵ are independently selected from the group of hydrogen, hydroxyl, an aliphatic or non-aliphatic straight- or branched-chain C1 to C10 hydrocarbon, alkoxy, aryloxy, nitro, cyano, chloro, fluoro, bromo, iodo, haloalkyl, dihaloalkyl, trihaloalkyl, amino, alkylamino, dialkylamino, acylamino, arylamino, amido, alkylamido, dialkylamido, arylamido, aryl, C5 to C7 cycloalkyl, and arylalkyl;
R¹⁰ is H(Z)N—, H(Z)N-hydrocarbon-, H(Z)N-hydrocarbon-N(Z)-hydrocarbon-, H(Z)N-hydrocarbon-N(Z)-hydrocarbon-N(Z)-hydrocarbon-, H(Z)N-hydrocarbon-O-hydrocarbon-, H(Z)N-hydrocarbon-O-hydrocarbon-N(Z)-hydrocarbon-, hydrocarbon-O-hydrocarbon-, hydrocarbon-N(Z)-hydrocarbon-, H(Z)N-hydrocarbon-carbonyl-hydrocarbon-, hydrocarbon-carbonyl-hydrocarbon-, H(Z)N-phenyl-, H(Z)N-phenylalkyl-, H(Z)N-phenylalkyl-N(Z)-hydrocarbon-, H(Z)N-phenylalkyl-N(Z)-hydrocarbon-N(Z)-hydrocarbon-, H(Z)N-phenylalkyl-O-hydrocarbon-, H(Z)N-phenylalkyl-O-hydrocarbon-N(Z)-hydrocarbon-, phenylalkyl-O-hydrocarbon-, phenylalkyl-N(Z)-hydrocarbon-, H(Z)N-phenylalkyl-carbonyl-hydrocarbon-, or phenylalkyl-carbonyl-hydrocarbon-, wherein each hydrocarbon is independently an aliphatic or non-aliphatic straight- or branched-chain C1 to C10 group, and wherein each alkyl is a C1 to C10 alkyl; and
Z is independently hydrogen or t-butoxycarbonyl.

2. The compound according to claim 1 wherein R² is selected from an aliphatic or non-aliphatic straight- or branched-chain C1 to C30 hydrocarbon, phenyl, phenylalkyl, substituted phenyl, and substituted phenylalkyl.

3. The compound according to claim 2 wherein R² is an aliphatic or non-aliphatic straight- or branched-chain C10 to C20 hydrocarbon.

4. The compound according to claim 2 wherein R² is an aliphatic or non-aliphatic straight- or branched-chain C14 to C16 alkyl.

5. The compound according to claim 1 wherein R² is a poly(alkyl)amine, poly(alkoxy)amine, or polyamine.

6. The compound according to claim 5 wherein R² is spermine.

7. The compound according to claim 1, wherein R² is a C10 to C20 alkyl group.

8. The compound according to claim 1, wherein R² is a C10 to C20 alkenyl group.

9. The compound according to claim 1, wherein the compound is selected from
(4R)-2-(benzo[d][1,3]dioxol-5-yl)-N-decylthiazolidine-4-carboxamide;
(4R)-2-(benzo[d][1,3]dioxol-5-yl)-N-dodecylthiazolidine-4-carboxamide;
(4R)-2-(benzo[d][1,3]dioxol-5-yl)-N-tetradecylthiazolidine-4-carboxamide;
(4R)-2-(benzo[d][1,3]dioxol-5-yl)-N-hexadecylthiazolidine-4-carboxamide;
and salts thereof.

10. A method of destroying a cancer cell comprising:
providing a compound according to claim 1; and
contacting the cancer cell with the compound under conditions effective to kill the cancer cell.

11. The method according to claim 10, wherein the cancer is selected from prostate cancer, breast cancer, ovarian cancer, and skin cancer.

12. The method according to claim 10, wherein the compound is selected from
(4R)-2-(benzo[d][1,3]dioxol-5-yl)-N-decylthiazolidine-4-carboxamide;
(4R)-2-(benzo[d][1,3]dioxol-5-yl)-N-dodecylthiazolidine-4-carboxamide;
(4R)-2-(benzo[d][1,3]dioxol-5-yl)-N-tetradecylthiazolidine-4-carboxamide;
(4R)-2-(benzo[d][1,3]dioxol-5-yl)-N-hexadecylthiazolidine-4-carboxamide;
and salts thereof.

13. A method of treating cancer comprising:
providing a compound according to claim 1; and
administering the compound to a patient having cancer, wherein said administering is effective to kill cancer cells and thereby treat the cancer.

14. The method according to claim 13, wherein said administering is carried out systemically.

15. The method according to claim 13, wherein said administering is carried out directly to a site where cancer cells are present.

16. The method according to claim 13, wherein said administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

17. The method according to claim 13, wherein the cancer is selected from prostate cancer, breast cancer, ovarian cancer, and skin cancer.

18. The method according to claim 17 wherein the skin cancer is malignant melanoma.

19. The method according to claim 17 wherein the skin cancer is non-malignant melanoma.

20. The method according to claim 13, wherein the compound is administered at a dosage rate of about 0.01 to about 100 mg/kg·body weight.

21. The method according to claim 13, wherein said administering is repeated periodically.

22. The method according to claim 13, wherein said administering is carried out in combination with another cancer therapy.

23. The method according to claim 13, wherein the compound is selected from
(4R)-2-(benzo[d][1,3]dioxol-5-yl)-N-decylthiazolidine-4-carboxamide;
(4R)-2-(benzo[d][1,3]dioxol-5-yl)-N-dodecylthiazolidine-4-carboxamide;
(4R)-2-(benzo[d][1,3]dioxol-5-yl)-N-tetradecylthiazolidine-4-carboxamide;
(4R)-2-(benzo[d][1,3]dioxol-5-yl)-N-hexadecylthiazolidine-4-carboxamide;
and salts thereof.

24. A method of making a compound according to claim 1 comprising:
providing a first intermediate compound having a formula 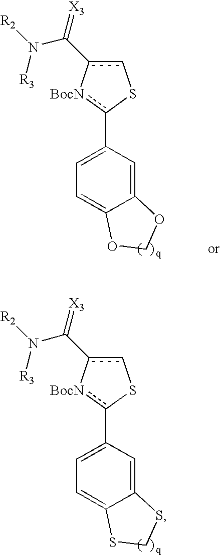
wherein Boc is a protective group; and
converting the first intermediate compound to the compound.

25. The method according to claim 24, wherein said providing the first intermediate compound comprises:
providing a second intermediate compound having a formula 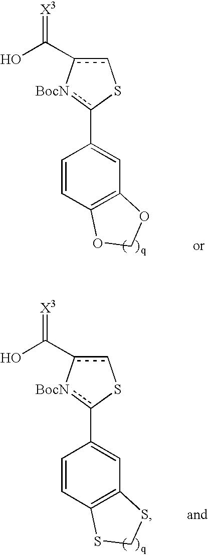
reacting the second intermediate compound with HNR₂R₃ under conditions effective to form the first intermediate compound.

26. The method according to claim 24, wherein said providing the second intermediate compound comprises:
reacting a compound having a formula 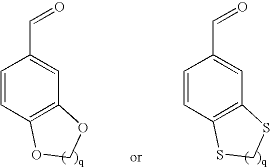
with a compound having a formula 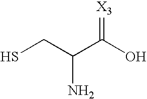
under conditions effective to form the second intermediate compound.
